(19) United States
(12) Patent Application Publication
(10) Pub. No.: US 2024/0427955 A1
(43) Pub. Date: Dec. 26, 2024

(54) INTELLIGENT SIMULATION DEVICE FOR BOTTOM SEDIMENT POLLUTION PROCESS AND CONTROL AS WELL AS EXPERIMENTAL METHOD

(71) Applicant: CHINESE RESEARCH ACADEMY OF ENVIRONMENTAL SCIENCES, Beijing (CN)

(72) Inventors: Xia JIANG, Beijing (CN); Zhihao WU, Beijing (CN); Shuhang WANG, Beijing (CN); Junyi CHEN, Beijing (CN); Kun WANG, Beijing (CN); Qing CAI, Beijing (CN)

(21) Appl. No.: 18/261,405

(22) PCT Filed: Mar. 7, 2023
(86) PCT No.: PCT/CN2023/080125

§ 371 Date: Jul. 13, 2023

(30) Foreign Application Priority Data
Mar. 14, 2022 (CN) 202210243865.5

(51) Int. Cl. G06F 30/20; G01N 33/18

(52) U.S. Cl. CPC G06F 30/20; G01N 33/188

(57) ABSTRACT

Disclosed are an intelligent simulation device for bottom sediment pollution process and control as well as an experimental method. The device includes an experimental flume to which a water inlet tank and a water return tank are connected; a wave-making system, an illumination system, a dosing system and an aeration system; an environmental condition parameter online monitor capable of determining water environmental parameters online; an online water quality index analyzer for monitoring water quality indexes in real time; a water automatic sampling device for automatically collecting water samples in a single or cycle mode; an offline analyzer capable of determining water quality indexes of the collected water samples or physical-chemical indexes of sediments; and an automatic control module for automatically controlling various devices. Further disclosed is an experimental method based on the intelligent simulation device for bottom sediment pollution process and control.

### CROSS-REFERENCE TO RELATED APPLICATION

This patent application is a national stage application of International Patent Application No. PCT/CN2023/080125, filed on Mar. 7, 2023, which claims the benefit and priority of Chinese Patent Application No. 202210243865.5, filed with the China National Intellectual Property Administration on Mar. 14, 2022, the disclosure of which is incorporated by reference herein in its entirety as part of the present application.

### TECHNICAL FIELD

The present relates to the technical field of bottom sediment pollution experiments, and in particular to an intelligent simulation device for bottom sediment pollution process and control as well as an experimental method.

### BACKGROUND

Lake eutrophication, especially the internal loading caused by the release of nitrogen and phosphorus from sediments, is the key concern of the researches for global lakes. The nutrients in water (ammonia nitrogen/nitrate nitrogen/phosphorus) enter the sediment layer through the complex biogeochemical processes, and are released to the water body under the change of environmental factors, thus becoming internal pollutants of the lake ecosystem, which may lead to eutrophication and algal blooms. There are internal loadings of nitrogen and phosphorus in sediments to varying degrees in many lakes in China (Lake Taihu, Lake Dianchi, Lake Erhai, etc.). Environmental parameters, hydrodynamics, illumination and other conditions of the sediment/water interface are important factors affecting the geochemical and dynamic transfer-release of nitrogen and phosphorus in sediments. Formation mechanisms of internal loadings of nitrogen and phosphorous in lake sediments under environmental conditions and assessment of release risk of nitrogen and phosphorus are the research focuses of lake eutrophic evolution research in China, which can also provide theoretical support for internal loading control and water ecological restoration of the lakes. Many types of lakes in China lead to the complex formation mechanisms of water environment conditions and internal loading, and thus it is necessary to develop a simulation device to simulate water environments, hydrodynamic conditions, physical-chemical properties of bottom sediments and water of different types of lakes, for example, anoxic layer of the hypolimnion and alkaline conditions (pH>9.0) in lakes favorable for phosphorus release; disturbance of wind and waves on the sediment/water interface, illumination, and the organic content and physical-chemical properties of nitrogen, phosphorous and heavy metals in sediment and water. The accurate measurement of the environmental conditions and pollutants at interface such as nitrogen and phosphorus for this simulation device is the premise and key to fully understand the internal loading and transfer-release of nitrogen and phosphorus in lake sediments.

The present devices for simulating environmental processes for pollutants at sediment/water interface are small devices with limited functions, including two types: (1) The first type of simulation device is single in function and can only simply simulate the transfer-release of nitrogen and phosphorus, polychlorinated biphenyls or metal elements in sediments. With reference to the following three patents: (i) “Water environment simulation reactor based on water-soil interface” (invention patent in China, with application number: 2011104126804); (ii) “Device for simulating polychlorinated biphenyl release at sediment-water interface (utility model patent, with application number: 2010202028901)”; and (iii) “Simulation device for measuring geochemical process action of river sediment-water micro-interface (invention patent, with application number: 2012100349456)”. (2) The second type of simulation device is relatively complex, which not only can simulate the environmental process, but also can sample and monitor the environmental parameters online. Among them, the most advanced devices are: (iv) “Device and method for simulating nitrogen source and fate in in-situ river ecosystem” (invention patent, with application number: ZL201610018892.7); (v) “Device for simulating anammox process at in-situ river bottom sediment, and use method and application thereof” (invention patent, with authorized patent number: ZL2014106334415); (vi) “Multifunctional device for simulating lake ecological restoration, and use method and application thereof” (invention patent, with authorized patent number: ZL201110004505.1); (vii) “Indoor simulation device applied to lake sediment-water interface process study” (utility model patent, with application number: 2007200439279).

In conclusion, the first type of device is simple in structure, including: a main device for placing sediments/water, a test probe for measuring environmental conditions, an illumination device or a stirring rod for resuspension, and a pollutant collection device. The second type of simulation devices has a relatively complete structure, including: a water inlet device, a main device (cylindrical or box) for simulating lakes or rivers, a static gas collection box or sampling port, an environmental parameter monitoring probe, a light source, and a control system for water temperature. The carriers for nitrogen cycling bacteria, submerged plants and sediments can also be placed in some main devices. In two of the above patents (iv and v), a computer is connected to the environmental parameter monitoring probe, so as to display pH/DO/temperature parameters of the sediment/water interface online.

The first type of device above is too simple and lacks automatic water sample collection and online water quality monitoring device, and the device needs to be manually controlled to operate due to the absence of a computer control system. For example, only one environmental parameter monitoring probe and a stirring rod for resuspension are installed to simulate limited environmental conditions (temperature), hydraulic conditions (interface resuspension caused by wind and waves) or illumination conditions. The second type of device can comprehensively regulate and optimize water temperature, dissolved oxygen and illumination, and is provided with sampling ports for water and sediment samples, a hydraulic disturbance device and an environmental parameter monitoring probe, and some devices are also provided with computers and displays. However, the second type of device cannot achieve automatic and accurate control and real-time monitoring of interface environmental parameters or water quality, that is, it cannot control a variety of water environmental conditions and cannot collect water samples automatically and analyze water quality indexes online. The computer of the existing device can only display environmental parameters, but cannot control the operation of various components and display change curves of the environmental parameters. The automatic operation of various subsystems and data feedback cannot be directed due to the absence of the intelligent control system.

In conclusion, the technical problems needing to be solved urgently in the existing devices include: (1) the function of regulating environmental condition parameters of the sediment/water interface is limited, and it is impossible to accurately control various environmental parameters of the interface. (2) The functions of detecting various water quality indexes and environmental parameters online and collecting water samples automatically are absent. (3) Due to the absence of intelligent control system, the simulation device is operated manually, leading to low efficiency; and inability to display process parameters in real time, monitor data change curves, and achieve the continuous operation for many days (more than 60 days) and the automatic save of real-time data.

Therefore, an intelligent simulation device for bottom sediment pollution process and control as well as an experimental method are provided to solve the problems in the prior art.

### SUMMARY

An objective of the present disclosure is to provide an intelligent simulation device for bottom sediment pollution process and control as well as an experimental method to solve the problems existing in the prior art. The device has the characteristics of automation, visualization, intelligence, modularization and the like, and can achieve the research and development and verification of multiple sediment pollution control technologies.

In order to achieve the above objective, the present disclosure provides the following solution:

It is provided an intelligent simulation device for bottom sediment pollution process and control according to the present disclosure. The device includes:
an experimental flume, which can be used for simulating the nitrogen and phosphorus process at a sediment/water interface, where a water inlet tank and a water return tank are connected to the experimental flume, the water inlet tank can be used to preserve experimental water and to add water to the experimental flume; the water return tank is connected to the experimental flume, and can be used to achieve the return between water bodies of the experimental flume and the water return tank;
a wave-making system, which is installed on an upper part of an inner wall of the experimental flume and can be used to produce wind waves for the sediment/water interface;
an illumination system, which is installed directly above the experimental flume and can be used to simulate illumination;
a dosing system, which is installed below the experimental flume and can be used to add experimental reagents into the experimental flume to control the pH of the water body in the experimental flume, or can be used to add pollutants or flocculants;
an aeration system, which is installed on the upper part of the inner wall of the experimental flume, and can be used to perform aeration treatment on the water body in the experimental flume, so as to control dissolved oxygen of the water body in the experimental flume;
an environmental condition parameter online monitor, which is installed on the inner wall of the experimental flume and can extend to different depths of the water body in the experimental flume for online determination of water body environmental parameters, such as the environmental parameters include pH, Eh, DO, and conductivity;
an online water quality index analyzer, which can be used to monitor water quality indexes in real time, where the water quality indexes include total nitrogen, ammonia nitrogen, total phosphorus, and COD_{Cr};
a water automatic sampling device, which is connected to the experimental flume or the water return tank, and can automatically collect water samples in a single or cycle mode;
an offline analyzer, which can be used to determine water quality indexes of the collected water samples or physical-chemical indexes of sediments; and
an automatic control module, where the wave-making system, the illumination system, the dosing system, the aeration system, the environmental condition parameter online monitor, the online water quality index analyzer, the water automatic sampling device and the offline analyzer are all connected to the automatic control module, and the above split devices are directed by the automatic control module to operate according to set parameters and collect data online.

Preferably, the experimental flume and the water inlet tank are both installed on a support, and a support cavity is provided below the support. The support cavity can be used to accommodate experimental devices. Each of the experimental devices includes a peristaltic pump, a valve, a pipeline, a water return tank, a solenoid valve, a sampling cup, and a tee. The experimental flume is connected to a water return tank through a return pipe, the experimental flume is further provided with an overflow weir, and the overflow weir is connected to the water return tank through an overflow pipe.

Preferably, four experimental flumes and four water inlet tanks are correspondingly provided. The four experimental flumes can operate independently. Alternatively, after the four experimental flumes are connected in series, the water body can circulate and run.

Preferably, a main device of the wave-making system is a wave maker, which is fixed onto the inner wall of the experimental flume and can be used for performing height adjustment in the surface water.

A main device of the illumination system is an illumination lamp, which has a color temperature of 400 k, a power of 150 W and an illumination intensity from ( ) Lux to 10,000 Lux. The illumination lamp can automatically simulate the sunlight change within 24 hours a day, and the illumination intensity of the illumination lamp can also be set to be fixed.

Preferably, the dosing system includes reagent bottles, the number of the reagent bottles is three, and the three reagent bottles are respectively used for placing acid, alkali and reagents. The reagent bottles each are connected to the water return tank through a dosing pipeline, and a metering pump is connected to the dosing pipeline.

The aeration system includes an aeration head, the aeration head is connected with a gas supply system through a gas path, and the gas path is also connected with an air pump. The gas path is provided with a flowmeter, a control valve, and a pressure relief valve.

Preferably, the environmental condition parameter online monitor includes monitoring probes. The monitoring probes include a pH electrode, a fluorescence dissolved oxygen sensor, a conductivity electrode, and an Eh electrode. The monitoring probes each are fixed onto the inner wall of the experimental flume, and the depth of the monitoring probe in the water body can be adjusted through a scale holder made of aluminum alloy.

Preferably, the online water quality index analyzer includes an ammonia nitrogen automatic analyzer, a total phosphorus/total nitrogen automatic analyzer, and a COD_{Cr} automatic analyzer, and the online water quality index analyzer is connected to the experimental flume.

Preferably, there are two water automatic sampling devices, the refrigerator type sampling instrument is used as the water automatic sampling device, and the water automatic sampling device can be connected to the water body in the water return tank or the experimental flume.

The offline analyzer includes a multifunctional microplate spectrophotometer, a GC/MS analyzer, an ICP-MS analyzer, a laser particle size analyzer, a Unisense microelectrode, and a planar optode system.

Preferably, a PLC (Programmable Logic Controller Control System) is used as the automatic control module, and the automatic control module is installed in a control cabinet. A touch screen page of an industrial personal computer of the automatic control module can be used to display the structure and operation status of the intelligent simulation device, set the operation parameters of the split devices, and display the online monitoring number and the change curves of environmental parameters, water quality indexes and other data along with time in real time.

It is provided an experimental method based on the intelligent simulation device for bottom sediment pollution process and control according to the present disclosure. The method includes the following steps:
Step one: collecting sediment and water samples;
Step two: adding the sediments to an experimental flume;
Step three: adding the water samples to the experimental flume;
Step four: turning on an automatic control module, and then a wave-making system and an illumination system;
Step five: turning on an environmental condition parameter online monitor for determination of environmental parameters in water body in real time;
Step six: turning on an online water quality index analyzer for real-time monitoring of water quality indexes;
Step seven: turning on a water automatic sampling device;
Step eight: controlling environmental conditions;
Step nine: starting a simulation experiment;
Step ten: after the simulation experiment is completed, stopping the experiment; draining water and discharging sediments; and
Step eleven: preparing for the next simulation experiment.

Compared with the prior art, the present disclosure has the following technical effects:

For the first time, subsystems (split devices), such as an experimental flume, a water inlet tank, a peristaltic pump, a submersible pump, a pipeline, a valve, an environmental condition control system, a lighting and wind wave system, a water body automatic sampling device, an environmental parameter online monitor (pH/DO/Eh/conductivity), and a water quality parameter online monitor, are connected and installed, and an intelligent control system is developed to automatically operate the above subsystems. The intelligent control system is configured to set operation parameters of the simulation device, and thus it can be used to (1) accurately control various environmental conditions of the sediment/water interface. (2) automatically test the environmental condition parameters and water quality indexes of the sediment/water interface, and (3) automatically collect water samples. An industrial personal computer can be used to display the structures and operation status of the split devices, to set the operation parameters of the split devices, to display the process parameters and data curves in real time, and to save the data. In addition, the simulation device can be loaded with the in-situ testing technologies (diffusive gradients in thin films, planar optodes or microelectrode devices), so as to achieve the accurate testing (ammonia nitrogen/nitrate nitrogen/phosphorus/heavy metal and environmental parameters: DO/pH) of the sediment/water interface with the high spatial resolution (about 100 μm).

According to the simulation device, the following objectives can be achieved: (1) the formation mechanism of internal loading of nitrogen and phosphorus in the sediments, the influence of environmental parameters on the transfer-transform of nitrogen and phosphorus, and the migration cycle of nitrogen and phosphorus in water-sediment-algae-aquatic macrophyte interface are accurately revealed; (2) the establishment of Ecological risk assessment criteria and control technology systems for bottom sediment pollutants, and the formulation of technical guidelines for internal loading control of the sediments and sediment quality benchmarks and thresholds; (3) the research and development and verification of sediment pollution control technologies (ecological restoration, sediment passivation and environmental protection dredging).

### BRIEF DESCRIPTION OF THE DRAWINGS

To describe the technical solutions in the embodiments of the present disclosure or in the prior art more clearly, the following briefly introduces the accompanying drawings required for describing the embodiments. Apparently, the accompanying drawings in the following description show merely some embodiments of the present disclosure, and those of ordinary skill in the art may still derive other drawings from these accompanying drawings without creative efforts.

FIG. 1A is a structure schematic diagram of main equipment of an intelligent simulation device for bottom sediment pollution process and control in accordance with the present disclosure;

FIG. 1B is an enlarged view of I in FIG. 1A;

FIG. 1C is a schematic layout diagram of main split devices of a simulation system in accordance with the present disclosure in a laboratory;

FIG. 1D is a structure schematic diagram of an overflow weir/overflow pipe/return pipe device in accordance with the present disclosure;

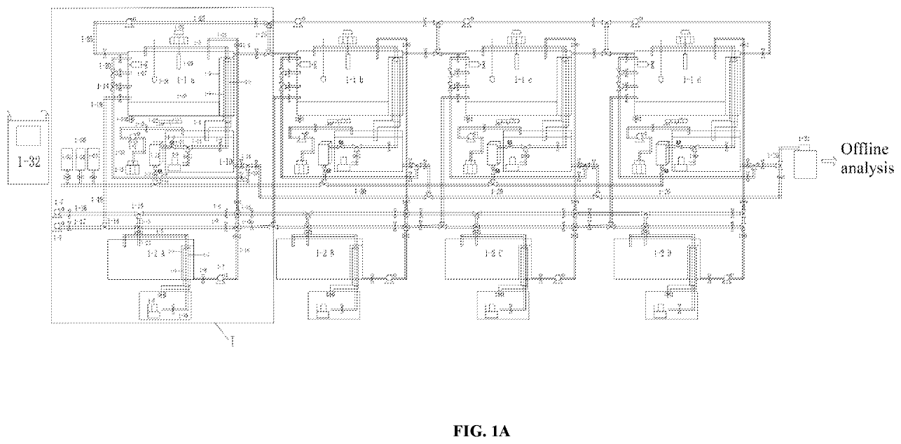

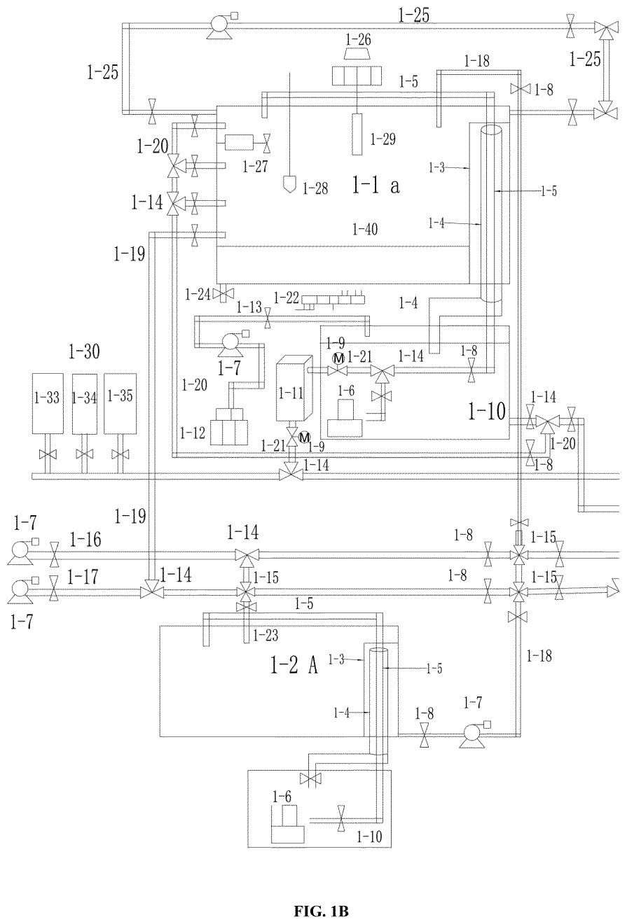

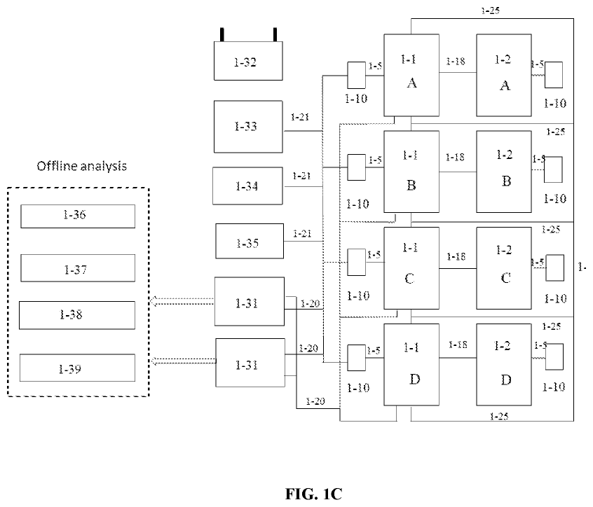

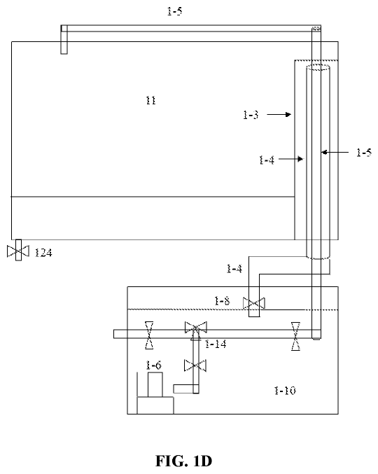

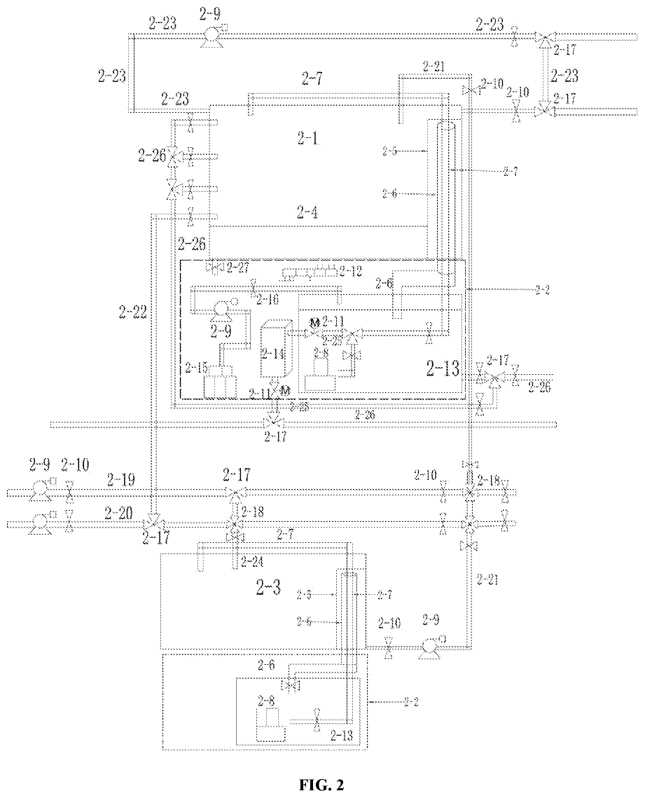

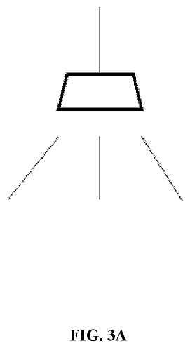

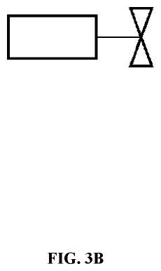

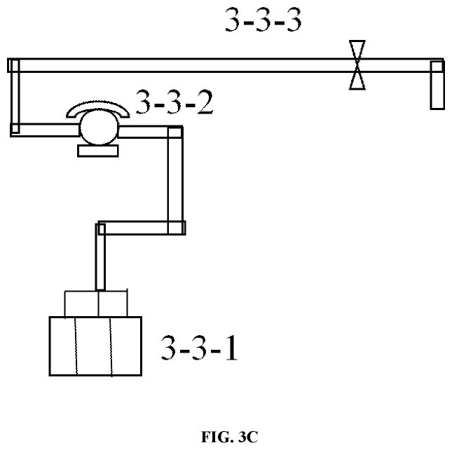

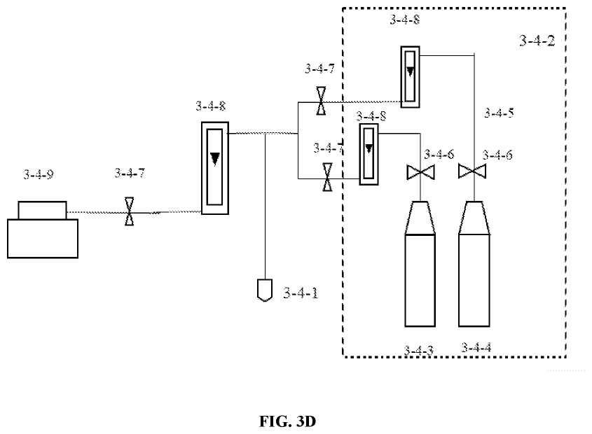

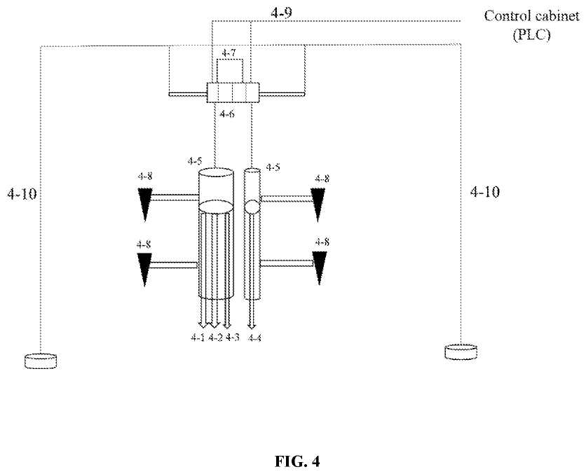

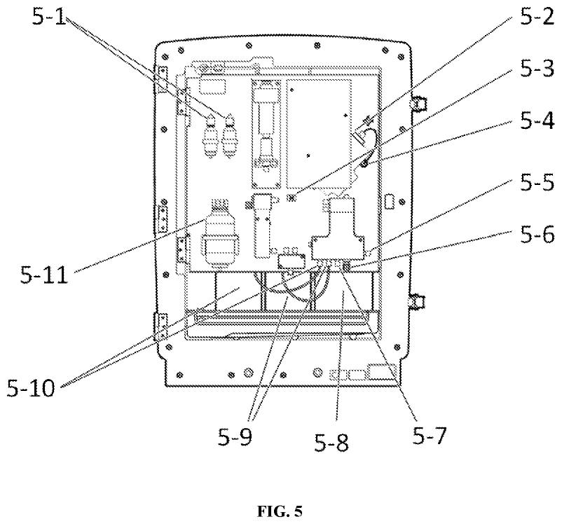

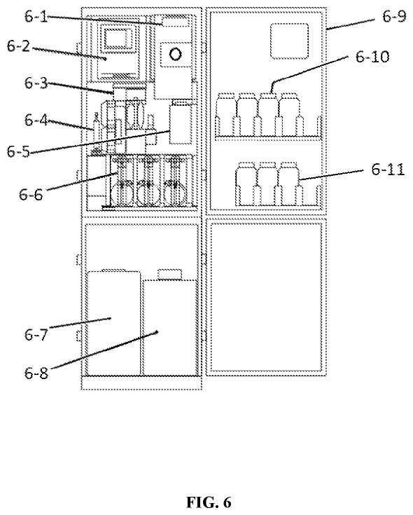

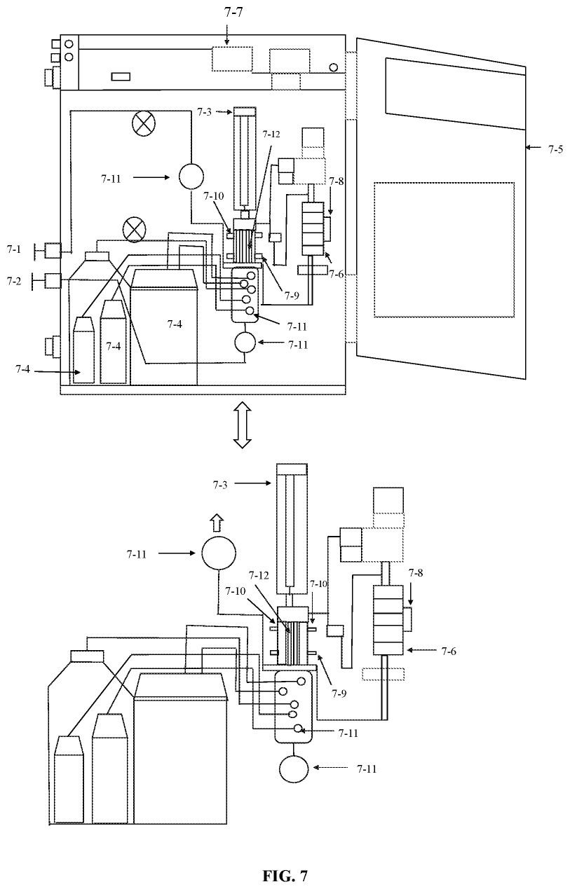

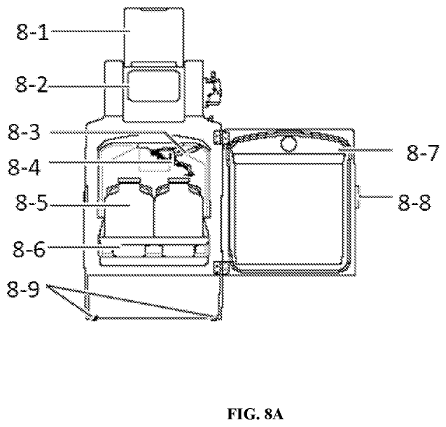

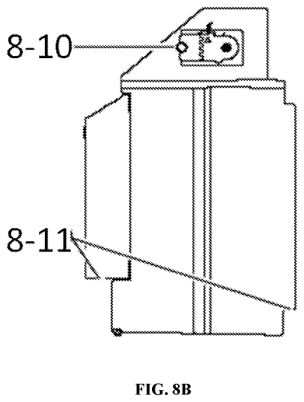

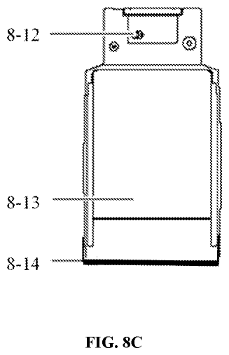

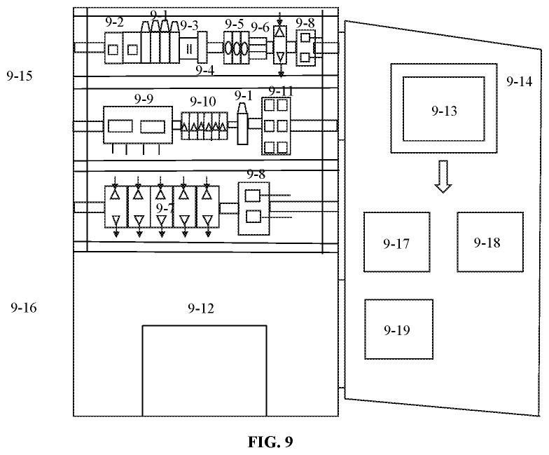

In FIG. 1A through FIG. 1D: 1-1—experimental flume (from left to right: a, b, c, d); 1-2—water inlet tank (from left to right: A, B, C and D); 1-3—overflow weir; 1-4—overflow pipe; 1-5—return pipe; 1-6—submersible pump; 1-7—peristaltic pump; 1-8—valve: 1-9—solenoid valve; 1-10—water return tank; 1-11—sampling cup; 1-12—dosing device; 1-13—water outlet pipe of dosing device; 1-14—tee; 1-15—cross; 1-16—main water inlet pipe; 1-17—main drainage pipe; 1-18—water inlet pipe; 1-19—drainage branch pipe; 1-20—water intake pipe of water automatic sampling device; 1-21—water inlet and outlet pipe of sampling cup; 1-22—PLC, integrated circuit, electric and electronic, and circuit board: 1-23—water inlet branch pipe; 1-24—sludge discharge pipe; 1-25—series pipeline; 1-26—illumination lamp; 1-27—wave-making device; 1-28—aeration head; 1-29—environmental condition parameter online monitor; 1-30—online water quality index analyzer; 1-31—water automatic sampling device; 1-32—automatic control module; 1-33—total phosphorus/total nitrogen automatic analyzer; 1-34—ammonia nitrogen automatic analyzer; 1-35—COD_{Cr} automatic analyzer; 1-36—GC/MS analyzer; 1-37—ICP-MS analyzer; 1-38—planar optode system (PO); 1-39—multifunctional microplate spectrophotometer; 1-40—sediment/water interface;

FIG. 2 is a schematic diagram of an experimental flume, a water inlet tank and auxiliary devices in accordance with the present disclosure;

In FIG. 2: 2-1—experimental flume; 2-2—support of experimental flume or water inlet tank; 2-3—water inlet tank; 2-4—sediment/water interface; 2-5—overflow weir; 2-6—overflow pipe; 2-7—return pipe; 2-8—submersible pump; 2-9—peristaltic pump; 2-10—valve; 2-11—solenoid valve; 2-12—PLC, integrated circuit, electric and electronic and circuit board; 2-13—water return tank; 2-14—sampling cup; 2-15—dosing device; 2-16—water outlet pipe of dosing device; 2-17—tee; 2-18—cross; 2-19—main water inlet pipe; 2-20—main drainage pipe; 2-21—water inlet pipe; 2-22—drainage branch pipe; 2-23—series pipeline; 2-24—water inlet branch pipe; 2-25—water inlet and outlet pipe of sampling cup; 2-26—water intake pipe of water automatic sampling device; 2-27—sludge discharge pipe;

FIG. 3A is a structure schematic diagram of an illumination system in accordance with the present disclosure;

FIG. 3B is a structure schematic diagram of a wave-making system in accordance with the present disclosure;

FIG. 3C is a structure schematic diagram of an automatic dosing device in accordance with the present disclosure;

FIG. 3D is a structure schematic diagram of an aeration device in accordance with the present disclosure;

In FIG. 3A through FIG. 3D: 3-1—illumination lamp (external connected to PLC); 3-2—wave maker (externally connected to PLC); 3-3-1—reagent bottles with acid, base or pollutant; 3-3-2—variable-frequency peristaltic metering pump; 3-3-3—water inlet pipe of dosing device; 3-4-1—aeration head; 3-4-2—centralized gas supply system (argon/pure oxygen) in laboratory; 3-4-3—steel argon cylinder; 3-4-4—steel pure oxygen cylinder; 3-4-5—gas path; 3-4-6—pressure relief valve; 3-4-7—control valve; 3-4-8—flowmeter: 3-4-9—air pump;

FIG. 4 is a structure schematic diagram of an environmental condition parameter online monitor in accordance with the present disclosure;

In FIG. 4: 4-1—Eh electrode; 4-2—conductivity electrode; 4-3—fluorescence dissolved oxygen sensor; 4-4—pH electrode; 4-5—electrode housing; 4-6—SC1000 multi-parameter universal controller; 4-7—multi-parameter wall-mounted meter head; 4-8—stainless steel sensor holder; 4-9—signal cable; 4-10—scale holder made of aluminum alloy;

FIG. 5 is a structure schematic diagram of an ammonia nitrogen automatic analyzer in accordance with the present disclosure;

In FIG. 5: 5-1—electrolyte replacement bottle; 5-2—electrode assembly; 5-3—membrane cap fixing piece; 5-4—electrode plate joint; 5-5—interface connected to test tube; 5-6—discharge interface; 5-7—sampling tube; 5-8—reagent; 5-9—standard solution (high standard); 5-10—standard solution (low standard); 5-11—cleaning solution;

FIG. 6 is a schematic diagram of a total phosphorus/total nitrogen automatic analyzer in accordance with the present disclosure;

In FIG. 6: 6-1—power switch and circuit board; 6-2—liquid crystal display screen and button; 6-3—heating decomposition system; 6-4—metering pump; 6-5—detector; 6-6—reagent pump; 6-7—pure water flume; 6-8—waste solution tank; 6-9—instrument door; 6-10—TN/TP standard solution and sodium hydroxide/hydrochloric acid reagent; 6-11—reagent; potassium persulfate/ascorbic acid/ammonium molybdate;

FIG. 7 is a schematic diagram of a COD_{Cr} automatic analyzer in accordance with the present disclosure;

In FIG. 7: 7-1—sample injection tube; 7-2—waste solution discharge pipe; 7-3—piston pump; 7-4—reagent bottle; 7-5—instrument door; 7-6—digestion unit; 7-7—liquid crystal display screen and button; 7-8—photometer; 7-9—low-liquid-level photometer; 7-10—high-liquid-level photometer; 7-11—valve unit; 7-12—metering tube;

FIG. 8A is a front view of a water automatic sampling device in accordance with the present disclosure;

FIG. 8B is a left view of a water automatic sampling device in accordance with the present disclosure;

FIG. 8C is a right view of a water automatic sampling device in accordance with the present disclosure;

In FIG. 8A through FIG. 8C: 8-1—control panel cover; 8-2—control panel; 8-3—discharge pipe; 8-4—distribution arm; 8-5—sampling bottle; 8-6—bottle rack; 8-7—refrigerator door; 8-8—bolt; 8-9—height adjustment rack; 8-10—liquid detector and pump; 8-11—support point; 8-12—connection of external facility; 8-13—refrigeration assembly; 8-14—alternating current main line;

FIG. 9 is a structure schematic diagram of a control cabinet in accordance with the present disclosure;

In FIG. 9: 9-1—switch; 9-2—electric leakage protector; 9-3—socket; 9-4—power supply; 9-5—switchboard; 9-6—fuse terminal; 9-7—wiring terminal; 9-8—data acquisition module; 9-9—PLC; 9-10—relay; 9-11—wireless bridge; 9-12—industrial personal computer host; 9-13—screen; 9-14—control cabinet door; 9-15—control cabinet upper part; 9-16—control cabinet lower part; 9-17—schematic diagram of screen device structure and parameter; 9-18—parameter setting and control page in computer screen; 9-19—page on screen for online display of change curves of environmental condition parameter and water quality index.

### DETAILED DESCRIPTION OF THE EMBODIMENTS

The following clearly and completely describes the technical solutions in the embodiments of the present disclosure with reference to the accompanying drawings in the embodiments of the present disclosure. Apparently, the described embodiments are merely a part rather than all of the embodiments of the present disclosure. All other embodiments obtained by a person of ordinary skill in the art based on the embodiments of the present disclosure without creative efforts shall fall within the protection scope of the present disclosure.

An objective of the present disclosure is to provide an intelligent simulation device for bottom sediment pollution process and control as well as an experimental method to solve the problems existing in the prior art. The device has the characteristics of automation, visualization, intelligence, modularization and the like, and can achieve simulation experiments of nitrogen and phosphorus transfer-release processes at the lake sediment/water interface and the research and development and verification of multiple control technologies for pollution in sediments.

To make the objectives, features and advantages of the present disclosure more apparently and understandably, the following further describes the present disclosure in detail with reference to the accompanying drawings and the specific embodiments.

### Embodiment 1

As shown in FIG. 1A through FIG. 9, it is provided an intelligent simulation device for bottom sediment pollution process and control according to an embodiment of the present disclosure. In the drawings, the reference numerals of the device are described as follows:
Peristaltic pump: [symbol]
Submersible pump: [symbol]
Valve: [symbol]
Solenoid valve: [symbol]
Tee valve: [symbol]
Cross valve: [symbol]
Sampling cup: [symbol]
Dosing device: [symbol]
PLC, integrated circuit, electric and electronic and circuit board:

[symbol]

The intelligent simulation device for bottom sediment pollution process and control of the embodiment can accurately control various environmental conditions of a sediment/water interface, monitor interface environmental parameters and water quality parameters in real time, and automatically collect water samples. According to the embodiment, automation, visualization, intelligence and modularization, real-time online monitoring of water environment parameters and water quality indexes of the water body in the experimental flume, and offline monitoring of sediments and water samples can be achieved. The expandability is strong, and the simulation of environmental process of pollutants at the sediment/water interface can be achieved through the simulation device.

In this embodiment, the simulation device is mainly formed by tightly combined installation of multiple subsystems (split devices) and pipes/pumps/valves, and can simulate various types of water environmental conditions (pH/DO/Eh/conductivity), hydrodynamic conditions (wind and waves) and illumination conditions for lake sediments and water bodies, thus achieving real-time monitoring of water environment parameters and water quality indexes, and automatic collection of water samples. An automatic control module with an industrial personal computer and a PLC (programmable logic controller) as the core is used to control the automatic operation of the device, to display testing data curves online, and to save the data. As shown in FIG. 1A through FIG. 1C, the structural functions and use methods of the split devices and pipelines/pumps/valves are as follows:

(1) Four water inlet tanks (volume: 1.20 m×0.80 m×1.00 m): The water inlet tanks are used to store collected lake water or water discharged from an experimental flume, where the water inlet tank is made of acrylic (thickness: 30 mm). The peristaltic pump is turned on, and then the water inlet and outlet operations between the water inlet tank and the experimental flume can be achieved through a rigid PVC (rigid polyvinyl chloride) pipe or a PU (polyurethane) hose. The experimental flume (volume: 1.20 m×0.80 m×1.20 m) is made of ultra-white glass (thickness: 12 mm), and is used to place sediments and water for performing the simulation experiments for environmental processes of nitrogen and phosphorus at a sediment/water interface. The wall of the experimental flume is transparent for observing the sediment/water interface and small devices in the experimental flume clearly. An illumination system is installed on the top of the experimental flume, and a wave-making system, an environmental condition parameter online monitor, a wave-making device and an aeration head of an aeration device are installed on the inner wall of the experimental flume. The experimental flume is internally provided with, or connected with, an overflow weir, an overflow pipe and a return pipe. The four experimental flumes may operate independently, or can be connected in series through pipelines and peristaltic pumps to achieve the continuous flow of water in the multiple experimental flumes.

The experimental flume and the water inlet tank each are placed on a support, the support is a cuboid with an opening at the lower part, which has a volume of 1.20 m×0.80 m×0.60 m, an upper plane wall thickness of 20 mm, and the peripheral side wall thickness of 5 mm; the interior of the support is a cavity, the support is made of carbon steel by plastic spraying, and the surface of the support is insulated and subjected to anticorrosive treatment. The support cavity is used to accommodate a series of small devices. The small devices in the support cavity of the experimental flume include a peristaltic pump, valves, various pipelines, a water return tank, solenoid valves, a sampling cup, a tee, a submersible pump, a dosing system, a circuit board, electric and electronic lines, and a signal cable. The small devices in the support cavity of the water inlet tank include a peristaltic pump, valves, pipelines, a water return tank, a submersible pump, a PLC, a circuit board, and electric and electronic lines.

The overflow weir and the return pipe inside the experimental flume (FIG. 1A through FIG. 3D) are used to achieve the water circulation between the experimental flume and the water return tank. A housing of the overflow weir (material: ultra-white glass) is a hollow cuboid with a height of 1.07 m, a wall thickness of 12 mm and a cross-sectional side length of 15 cm. The overflow weir is formed by bonding two planes in the right corner of the experimental flume and two strip-shaped ultra-white glass plates, and the bottoms of the two strip-shaped glass plates are glued to the bottom of the experimental flume. A vertical overflow pipe (material: rigid PVC pipe) is installed right in the middle of the housing of the overflow weir, with a total height of 1.15 m, an inner diameter of 3 cm, and the height above the bottom of the experimental flume of 1.06 m. The lower part of the overflow pipe passes through the bottom of the experimental flume, and the tail end is connected with a PVC corrugated hose (length: 9 cm, inner diameter: 4 cm). If the liquid level in the experimental flume exceeds the overflow pipe, the liquid can be introduced into the water return tank through such a pipe. The length, width and height of the water return tank are 50 cmx 40 cmx 30 cm, and the water return tank is made of acrylic. The return pipe is a 90-degree bent pipe, which is made of Rigid PVC pipe, has an inner diameter of 1.5 cm, a horizontal length of 1.00 m, and a vertical height of 1.30 m. The return pipe at the vertical part is installed right in the middle of the overflow pipe, and is concentrically sleeved with the overflow pipe. After the return pipe in the horizontal direction crosses the top end of the housing of the overflow weir, the top end of the return pipe extends to the left side of the experimental flume. The bottom end of the return pipe at the vertical part passes through the bottom of the experimental flume, and then is connected to a PU hose (inner diameter: 2 cm; length: 5 cm) and a reducer pipe (two opening inner diameters: 1.5 cm or 2 cm, length: 2 cm), and the other end of the PU hose is connected to an opening of a tee (material: rigid PVC). The submersible pump of the water return tank is connected to the tee through another PU hose (inner diameter: 2 cm; length: 5 cm). Water can be introduced into the left top end of the experimental flume through the opening of the tee, the PU hose and the return pipe, thus achieving the water circulation between the experimental flume and the water return tank. Another opening of the tee of the submersible pump is connected to a sampling cup interface of an online water quality index analyzer through a PU hose (inner diameter: 2 cm; length: 12 cm) for water intake of the online water quality index analyzer. A rigid water intake pipe (PVC) (inner diameter: 2 cm) of the water automatic sampling device is connected to one end of a section of PU hose (inner diameter: 2 cm; length: 5 cm), while an opening at the other end of this section of PU hose is fixed under the liquid level of the water return tank, so as to achieve the water intake of the water automatic sampling device. In addition, a rigid PVC pipe and three connected branches (water intake pipes of the water automatic sampling device) fixedly installed on the left side of the experimental flume are fixed to the surface layer, middle layer and bottom layer of the left inner wall of the experimental flume. The lower part of the rigid PVC pipe is bent to go across the bottom of the experimental flume, and the other end of the rigid PVC pipe is connected to a rigid pipe of an automatic sampling for sampling the water of the water automatic sampling device, thus sampling the surface water, middle water and bottom water. The bottom of the left side of the experimental flume is also provided with a sludge discharge pipe and a valve. The sludge discharge pipe is a PVC corrugated hose (inner diameter: 4 cm; length: 12 cm), such a sludge discharge pipe is provided with a valve, which is closed in general, and is opened for bottom sediment discharge after the experiment is finished.

The small devices in the support cavity below the water inlet tank include a peristaltic pump, a water return tank, a submersible pump, pipelines, a PLC, a circuit board, and electric and electronic lines. The small devices are completely the same as those below the experimental flume in dimensions, materials and functions. The water in the water inlet tank can be introduced into the experimental flume through the peristaltic pump, the water inlet pipe (material: rigid PVC pipe, inner diameter: 2.0 cm) and a valve. After the experiment is finished, the water in the experimental flume can be introduced into the water inlet tank for preservation through a drainage branch pipe, a main drainage pipe, a water inlet branch and valves. The water in the experimental flume can also be drained into the sewer through the drainage branch pipe, the main drainage pipe and the valves. The water in the water inlet tank can be drained into the sewer through the peristaltic pump, the valve, the water inlet pipe and the main drainage pipe. The main water inlet pipe, the water inlet branch pipe, the peristaltic pump and the valve can also be used to feed tap water or experimental water outside the laboratory into the water inlet tank for preservation, and then feed the water into the experimental flume through the water inlet pipe, the peristaltic pump and the valve. The main water inlet pipe, the water inlet pipe, the peristaltic pump and the valve can also be used to directly feed the tap water outside the laboratory into the experimental flume, and pollutant or reagents can be added by the dosing system to prepare simulated sewage. The water inlet and drainage for the experimental flume or the water inlet tank can also be completed by a PU hose (inner diameter: 3.0 cm; length: 3.0 m) and the peristaltic pump.

An environmental condition parameter online monitor, a wave-making system (wave maker) and an aeration system are fixedly installed on the upper side wall inside the experimental flume. An illumination system is installed at 1.0 m above the center of the experimental flume, and a dosing system is installed in the support cavity below the experimental flume. (i) The environmental condition parameter online monitor is used to monitor pH/DO/Eh/conductivity of a water body in real time (Hach Company. United States), including: pH, DO, Eh and conductivity sensors. Above sensor probes are fixed to the wall of the experimental flume by a stainless-steel sensor holder, and the depths of the sensor probes in the water can be adjusted, so as to determine environmental condition parameters of the surface, middle and bottom water online. (ii) The wave maker is fixed onto the wall of the experimental flume, and can be used to perform height adjustment in the surface water (0 cm to 30 cm), thus simulating wind and wave effect, and making the resuspension of surface sediments. (iii) An aeration head and an aeration pump are connected to a pure oxygen and argon gas pipeline, the air, pure oxygen or argon can be aerated through the aeration head, and the aeration depth can be adjusted in the surface and middle water. (iv) An illumination lamp can automatically simulate the change of sunlight within 24 hours a day, and the illumination intensity is from 0 Lux to 10.000 Lux. (v) The dosing system includes reagent bottles, a variable-frequency peristaltic metering pump, a PLC module, and a liquid adding pipeline. A wiring terminal, a data acquisition module, a PLC and a relay inside the control cabinet are connected to the dosing system, and the industrial personal computer is configured to set operating parameters and to control the dosing system to supply acid, alkali or reagent to the water return tank.

(2) Wave-making and illumination systems: The wave-making and illumination systems are used to create wind waves on the sediment/water interface and sunlight above the experimental flume. The schematic diagram of wave making and illumination of the illumination system are shown in FIG. 3. A wave maker (model number: 5000; Eheim Company, Germany; power: 5 W; frequency: 50 HZ) is fixed to the wall of the experimental flume, and can be used to perform height adjustment in the surface layer (0 cm to 30 cm), so as to simulate the wind and wave effect and make the resuspension of the surface sediments. The illumination lamp (LED) (color temperature: 400 k; power: 150 W; Guangzhou Maiguang Electronic Science Technology Co., Ltd.) is close to the spectral performance of the sun, and can automatically simulate the change of sunlight within 24 hours a day. The illumination intensity varies from 0 Lux to 10,000 Lux, and the illumination intensity of the illumination lamp can be fixed. An industrial personal computer of the control cabinet can be used to set the operating parameters, to control the operation of the wave-making and illumination systems, and to display online parameters of the illumination system on a display screen.

(3) Automatic dosing and aeration devices: a dosing module (FIG. 3) is also arranged under the support of the experimental flume, which is used to control the environmental parameters of pH and DO of water body, or to add pollutants or flocculants. The dosing module includes three reagent bottles (volume: 2 L) for placing acid, base or reagents, a variable-frequency peristaltic metering pump (model: BW-100; Baoding Chuangrui Precision Pump Co., Ltd., Hebei Province, China), a PLC module, and three liquid adding pipelines made of FEP (perfluoroethylene-propylene copolymer). One end of the liquid adding pipeline is connected to a metering pump, and the other end of the liquid adding pipeline is placed in the water return tank. The industrial personal computer of the control cabinet is used to set target parameter values (pH value of the experimental flume or the dosing amount), and the variable-frequency peristaltic metering pump is controlled by the PLC to add acid, alkali or reagents to the experimental flume at a certain flow rate (0 ml/min to 10 ml/min), and the dosing is automatically stopped until the pH in water in the experimental flume tested by the online monitoring module reaches the set target value or the dosing amount is reached. The automatic dosing module is connected with the control cabinet, and a target pH value is set in advance. According to the set target pH value and the pH fed back by the online monitor, the on/off and flow rate of the variable-frequency peristaltic metering pump are controlled. When the pH in water in the experimental flume or the addition of pollutants reaches the target value, the addition of acid, alkali or reagent is automatically stopped. The aeration device includes an air pump and an aeration head (model: 200-3702, Eheim Company, Germany), a laboratory centralized gas supply system (argon/pure oxygen), an argon/pure oxygen steel cylinder, a gas path (stainless steel pipe; BA class), a flowmeter, a control valve, a pressure relief valve, etc. The air pump has the maximum exhaust volume of 400 L/min and the power of 3.5 W, and is used for the water body in the experimental flume. A disc type aeration head is made of ethylene propylene rubber (EPDM). The PLC and frequency converter of the aeration device are connected to the control cabinet; and according to a DO target value set by the industrial personal computer and the data fed back by the online monitor, the on/off and flow rate of aeration are controlled, and the aeration is stopped when the target value is reached. The operation status and parameters of the automatic dosing and aeration devices are automatically displayed on the screen of the industrial personal computer.

(4) Environmental condition parameter online monitor: The environmental condition parameter online monitor is used to monitor pH/DO/Eh/conductivity of a water body in real time, including monitoring probes (FIG. 4) for determining water environmental parameters (pH/Eh/DO/salinity) online, including: a pH electrode (model: PD1R1; Hach, USA), a fluorescence dissolved oxygen sensor (model: LDOII; Hach, USA), a conductivity electrode (model: 3725E2T; Hach, USA), an Eh electrode (model: HQ30D; Hach, USA), a SC1000 multi-parameter universal controller, a multi-parameter wall-mounted meter, a stainless steel sensor holder, and a scale holder made of aluminum alloy. The measurement ranges of the four parameters are as follows: pH: 1-14; DO; 0 mg/L-20 mg/L; Eh: (integrated with a platinum electrode and an Ag—AgCl reference electrode): −1999 my-1999 mv; and conductivity: 0 μs/cm-2000.000 μs/cm. The sensor probes are fixed to the wall of the experimental flume, and their depths in the water are adjusted by the scale holder made of aluminum alloy, so as to determine the environmental parameters of surface, middle and bottom water online, with a vertical spatial resolution of 1.0 cm. Online monitored environmental parameters data are input into the control cabinet (PLC), the industrial personal computer can save the data in real time and display change curves of the four environmental parameters along with the time online. Meanwhile, the four environmental parameters are also automatically displayed on the screen of the industrial personal computer.

(5) Online water quality index analyzer: The Online water quality index analyzer is used to monitor water quality indexes such as total nitrogen, total phosphorus, ammonium nitrogen and COD_{Cr} in real time, the online test data can be transmitted to the industrial personal computer of the control cabinet, and the industrial personal computer can save the data in real time and display change curves of the above parameters along with time online. Meanwhile, the parameters are automatically displayed on the analysis image position of the screen of the industrial personal computer. The water quality analyzer is purchased from the products of Hach international brand, including three monitoring devices: an ammonia nitrogen monitoring device (Hach, USA, Amtax sc100), a total phosphorus/total nitrogen monitoring device (Hach, USA. NPW160H) and a COD_{Cr} automatic monitor (Hach, USA, CODmax plus sc), thus achieving real-time and accurate detection and analysis of ammonia nitrogen, total phosphorus, total nitrogen and COD_{Cr}. A sample injection port is provided in the water return tank of the experimental flume, and the submersible pump is used to provide power. A water sample is introduced into the online water quality index analyzer through water inlet and outlet pipes of the sampling cup. A solenoid valve is programmed and controlled by a control system of the control cabinet, and thus making the water sample entering the online analyzer have typicality and real-time performance. An intelligent control system of the control cabinet (PLC and industrial personal computer) is used to control the water quality index analyzer, thus achieving single determination (only analysis once) and cycle determination (continuous analysis of two to six times a day) for the four experimental flumes.

(i) The ammonia nitrogen automatic analyzer (as shown in FIG. 5) is used for rapid determination of ammonia nitrogen using an ammonia gas sensing electrode method, with the test range from 0-5 mg/L to 500 mg/L, the lowest detection limit of 0.02 mg/L and high precision. The ammonia nitrogen automatic analyzer has the functions of automatic cleaning, automatic calibration and automatic diagnosis, and small maintenance workload. Data transmission: the sc1000 multi-parameter universal controller is provided with a data cable. The modules include: a housing, a compressor, a collecting disc, a humidity sensor, an electrode assembly (an electrode body, an electrode, a sealing cover, a membrane cap, a joint), an electrolyte replacement bottle, a sampling tube, an overflow vessel, a filter, an air tube, a reagent, standard solution (high/low standard), cleaning solution, a reagent pump (valve pump), a pump head piston pump (10 ml), a discharge pipe, a heating discharge pipe, a FILTRX sampling tube, a discharge pipe, etc.

(ii) The digestion of the total phosphorus/total nitrogen automatic analyzer (as shown in FIG. 6) is based on GB11893-89: potassium persulfate is used as oxidant, water samples are digested at 120° C. for 30 min. phosphide is converted into phosphate ions, and molybdenum blue absorptiometry is used for detection (measuring wavelength: 700 nm); total nitrogen: the potassium persulfate is used as oxidant (according to GB11893-89): the water samples are digested at 120° C. for 30 min using the potassium persulfate as the oxidant; the nitride is converted into nitrate ion, and the pH of the sample solution is adjusted to 2 to 3; an ultraviolet absorptiometry is used for detection (measuring wavelengths: 220 nm and 275 nm; turbidity correction: A=A220−A275×2). The measurement range of total phosphorus or total nitrogen is 0.00 to 0.5/20.00/50.00/100.00 mg/L or 0.00 to 2.00/50.00/100.00/200.00 mg/L; and the minimum detection limit is 0.01 mg/L. Analysis units of the total phosphorous/total nitrogen include a digestion unit, a detector (ultraviolet-visible spectrophotometer), a valve unit, a display metering pump, a metering tube, a reagent pump, a heating decomposition device, a liquid tank for measurement range calibration, a reagent reservoir, a solution tank, a pure water flume, a waste solution tank, pure water, reagents (potassium persulfate, sodium hydroxide, hydrochloric acid, ammonium molybdate, ascorbic acid) and standard solution (TN/TP). In addition, there is an operation panel and a display screen.

(iii) A potassium dichromate method for COD_{Cr} automatic analyzer (as shown in FIG. 7) is used for COD_{Cr} determination, the principle for COD_{Cr} automatic analyzer is demonstrated as following: the mixed solution of a water sample, potassium dichromate, silver sulfate solution (catalyst makes the oxidation of straight-chain aromatic hydrocarbon compound more sufficiently) and concentrated sulfuric acid is heated to 175° C. in a digestion cuvette. During the period, the color of the chromium ion is changed as the chromium ion is reduced from VI valence to III valence as the oxidant, the degree of color change corresponds to the content of organic compounds in the sample. The COD_{Cr} of the sample is directly displayed by an analyzer through colorimetric conversion. The main interfering substance in COD_{Cr} detection of wastewater by COD_{Cr} rapid detector using CODmax II potassium dichromate is chloride, which can be removed by adding mercury sulfate to form a complex. The measuring range of the COD_{Cr} automatic analyzer is from 10 mg/L to 5.000 mg/L. The analysis units include a sample injection tube, a piston pump, a digestion unit, a spectrophotometer, a high-liquid-level photometer, a low-liquid-level photometer, a valve unit, a metering tube and reagents (potassium dichromate solution, mercury sulfate solution and sulfuric acid), and standard solution. In addition, there is an operation panel and a display screen.

The water sample collection of the above three monitoring equipment adopts the equipment piping system, and the industrial personal computer of the control cabinet is used to set the sampling time and frequency, etc. Through the submersible pump, the solenoid valve and the sampling cup near the water return tank, the samples are automatically collected from four experimental flumes and then are input into the above three automatic monitors for measuring the water quality indexes. Each of the three automatic monitors has an operation interface, and a manual menu interface can be executed to achieve functions such as parameter setting, correction, cleaning, operation, management and alarm; and the automatic operation can also be achieved through field bus control. That is, the monitors are connected to the intelligent control system (PLC and data reader) of the industrial personal computer in the control cabinet through a 4-core cables and RS485/RS232 interfaces, and the test parameters are set on the industrial personal computer, so as to control the automatic operation of the monitors. The industrial personal computer is used to set determination time, single or cycle test, etc, of one or more experimental flumes, then the three automatic monitors are triggered to operate, so as to feed the test data back to the industrial personal computer in real time for saving. The four parameters, and change curves of water quality indexes along with the time are displayed online on the display screen.

(6) Water automatic sampling device: A total of two water automatic sampling devices are provided, and an Isco 4700 refrigerator sampler (Teledyne Isco 4700, USA) is adopted for each device, as shown in FIG. 8. The full-automatic water sampler can be controlled and programmed to make the sampling fast, simple and easy. A controller can be used to flexibly control and display the sampling temperature, and to preserve twenty-four water samples at 4° C. A peristaltic pump is used to generate negative pressure to collect samples, and the samples are distributed to sampling bottles through the controller. The automatic sampling devices include: a suction pipe-linear FEP (perfluoroethylene-propylene copolymer), a peristaltic sampling pump, a sampling pipe, a liquid detector, a distribution arm, a pump filter, a controller, and sample bottles (twenty-four IL polyethylene bottles, where an automatic compressor is used to control the preservation temperature of sample bottles). The water quality automatic sampling device is connected to the PLC and data acquisition module of the control cabinet through a 4-core cable and a RS485 interface. The sampling frequency and volume can be set on the industrial control machine, thus sampling the water from the experimental flume or the water return tank, single sampling or cycle sampling can be conducted. The automatic sampling device operates automatically to take out the water samples and place the water samples temporarily in the refrigerator of the water sampler. A solenoid valve and the water quality automatic sampling device can be programmed by the control system to achieve single sampling (only once) and cycle sampling (up to six times a day).

(7) Offline analyzer: The offline analyzers include: instruments for analysis of heavy metals, nitrogen, phosphorus and organic matters: a SpectraMaxM2/M2e multifunctional microplate spectrophotometer, an Agilent GC/MS instrument; an Agilen ICP-MS; a Sympatec laser particle size analyzer for sediment particle size determination; a Danish Unisense microelectrode for determination of micro-interface environmental conditions of sediments, and a planar optode system (PO).

(8) Pipelines, valves and pumps: The pipelines are used to connect various experimental flumes, the water inlet tank, the water return tank, the online water quality index analyzer and the water automatic sampling device, so as to control water inlet, drainage, overflow, return and water intake. The pipelines of the device include cylindrical rigid PVC (rigid polyvinyl chloride) pipes. PU (polyurethane) hoses, and PVC corrugated hoses.

The return pipe and overflow pipe in the support cavity below the experimental flume are rigid PVC pipes (inner diameter: 1.5 cm or 3.0 cm), and the overflow pipe is connected to a reducer pipe below the bottom of the experimental flume (two openings: 3.0 cm or 4.0 cm respectively, and length: 3 cm) and a PVC corrugated hose (length: 9 cm, inner diameter: 4 cm). A water outlet of the submersible pump is connected to a section of PU hose (inner diameter: 2 cm; length: 5 cm), with a valve installed thereon (Burkert, Germany). The tail end of the section of PU hose is connected to a tee (material: rigid PVC), and the two openings of the tee are respectively connected to two PU hoses (inner diameter: 2 cm), the two PU hoses are respectively connected to a reducer pipe (inner diameters of two openings: 1.5 cm or 2 cm, length: 2 cm) at the sampling cup inlet of the online water quality tester or the return pipe, respectively. A sludge discharge pipe at the bottom of the experimental flume is a PVC corrugated hose (inner diameter: 4 cm; length: 12 cm), with a valve installed thereon. The main drainage pipe, the main water inlet pipe, the water inlet pipe, the drainage branch pipe, the water inlet branch pipe, series pipelines, the water outlet pipe of the sampling cup, the water intake pipe of the water automatic sampling device and the series pipelines are all rigid PVC pipes (inner diameter: 2.0 cm). A cross is provided at the connection of the main water inlet pipe and the water inlet pipe, and the water inlet and outlet directions are controlled by nearby valves. The region that the rigid PVC pipe of the main drainage pipe, the main water inlet pipe, the water inlet pipe and the series pipelines is close to the peristaltic pump is a section of PU hose (inner diameter: 2 cm, length: 15 cm). The middle part of the PU hose is arranged in a groove of the peristaltic pump, and both ends of the PU hose are connected to the interfaces of the rigid PVC pipe. The drainage branch and the water inlet branch pipes are all rigid PVC pipes and are not connected to the PU hose or the peristaltic pump.

The sampling cup below the experimental flume is a cube (5 cm×5 cm×10 cm), and two solenoid valves (model: CWX-25S; 24V DN20) copper; power: 5 W; Shanghai Xinjiao Valve Co., Ltd., China) are installed to control the water inlet and outlet of the sampling cup. The water outlet pipe below the sampling cup is a rigid PVC pipe (inner diameter: 0.5 cm; length: 2 m), which is connected to the online water quality index analyzer. A water inlet of the sampling cup is connected to a section of PU hose (inner diameter: 2 cm; length: 12 cm), which is connected to an opening of an outlet tee of the submersible pump. The water in the water return tank is fed by the submersible pump into the sampling cup through the tee, the PU hose and the solenoid valve, and then is fed into the online water quality index analyzer through another solenoid valve and the water outlet pipe (rigid PVC pipe). A drainage pipe is also installed on the side face of the sampling cup (material: rigid PVC pipe; diameter: 0.5 cm; length: 0.5 m), which is used to drain the residual solution from the sampling cup to the sewer. The two solenoid valves are controlled by programs of the industrial personal computer. When cleaning the sampling cup, only the valve for water outlet is opened, and the residual water of the sampling cup is drained from the drainage pipe; and then, another valve is opened, the water is fed from the submersible pump into the water inlet and outlet pipes and the sampling cup, and then flows into the online water quality analyzer.

For water inlet or drainage of the experimental flume, the PU hose (inner diameter: 2.0 cm, length: 3.0 m) can be used for manual operation; a part of the middle of the PU hose (length: 12 cm) is placed into the groove of the peristaltic pump, one end of the PU hose is placed into the experimental flume, and the other end of the PU hose is connected to the water inlet tank or a bucket to achieve the water inlet operation of the experimental flume. Alternatively, the other end of PU hose is placed in the sewer to drain water directly. In addition to the above pipeline valves (Burkert. Germany) and solenoid valves, elbows, tees, crosses, reducer pipes, choke plugs and other parts are also provided for the connection, turning, variable diameter, sealing, etc, of the above pipelines.

Two types of pumps are installed under the support of the experimental flume to provide kinetic energy and transport water and sediments for the whole experimental flume. (1) Two variable-frequency peristaltic metering pumps (model: BW-100 or CT1000; Baoding Chuangrui Precision Pump Co., Ltd., Hebei Province. China) have the flow rates from 0.04 ml/min to 36 ml/min or from 0.4 ml/min to 360 ml/min. respectively; the two types of pumps are used for the (i) the automatic dosing module or (ii) water inlet/drainage of the experimental flumes and the water inlet tank, series connection or sludge discharge of the experimental flumes. (2) One submersible pump (model: HQB-4500; power: 100 W; flow rate: 0.4 ml/min to 75 ml/min. Zhejiang Sensen Industrial Co., Ltd., China) is placed in the water return tank, which not only can return the water from the water return tank to the experimental flume, but also can introduce water to the water inlet and outlet pipes of the online water quality tester. The wave-making device is installed on the upper side face of the experimental flume, and a main device of the wave-making device is a wave maker (model: StreamON+5000; Eheim, Germany), with the maximum power of 800 w and a frequency of 50 Hz.

(9) Automatic control module: The automatic control module is an intelligent management system, which is installed in the control cabinet (as shown in FIG. 9) and consists of a PLC (Programmable Logic Controller), an industrial personal computer (industrial personal computer), a display screen, a switch, a data acquisition block (data reader), intelligent management software, etc. A PLC control system is used as the automatic control module, and a man-machine interface is provided. Through a data acquisition block, the functions of data communication, data acquisition and processing, parameter setting and online display of various parameters and data changes of the sensors, small devices and detection apparatus are achieved. The sensor is configured to output a detection signal to the PLC module, and the opening degree of the pump valves and the operation of various devices are programmed and controlled by the PLC, so as to control the opening/closing and flow rate of some pipelines, wave making, illumination, automatic dosing, aeration, water quality automatic sampling, water quality index online analysis, and real-time data collection from the environmental condition parameter online monitor and the above devices. Various subsystems each have complete independent control function and do not interfere with each other. The intelligent management software has the following functions: data acquisition; process parameters setting, data saving, real-time display of data and change curves of parameters along with time, identification, and remote control. A touch screen page of the industrial personal computer can be used to display the main device structures, to set parameters, and to display online monitoring data, change curves of environmental parameters and water quality indexes along with time in real time.

### (10) Use Method of Simulation Device

The start-up steps of “the intelligent simulation device for bottom sediment pollution process and control” are as follows: adding sediments→adding water→turning on wave-making system→turning on illumination system→turning on environmental condition parameter analyzer→turning on water quality index analyzer→turning on water automatic sampling device→controlling environmental conditions (aeration or dosing)→performing simulation experiment (including: online analysis of environmental condition parameters/water quality indexes, offline analysis of water samples, collection and testing of sediment samples, etc.)→stopping experiment→drainage→sludge discharge→preparation for the next simulation experiment. Because the simulation device has four experimental flumes and corresponding water inlet tanks, in general, the simulation experiments of four experimental flumes can be conducted in parallel, or only the simulation experiment of one experimental flume is conducted, and the experimental method is basically the same. If there are special experimental requirements, the series pipelines and valves can be opened and the peristaltic pump can be turned on to complete the series flow and closed return of the surface water in more than two experimental flumes.

In this embodiment, the simulation experiment corresponding to one experimental flume includes the following steps:

Adding of sediments: Barreled sediment and water mixture can be injected into the experimental flume using a peristaltic pump (model: CT1000; Baoding Chuangrui Precision Pump Co., Ltd., Hebei Province. China) and a PU hose (inner diameter: 4 cm; length: 2.0 m). The volume of a plastic barrel for containing sediments is 50 L. The barreled sediments can also be poured into the experimental flume directly from the upper part of the experimental flume through a guide plate. Finally, the sediments at the bottom of the experimental flume area are flattened. If there are aquatic plant vegetation restoration experiments, aquatic plant seedlings can be planted in the sediments.

Adding of water: Experimental water in the water inlet tank can be fed into the experimental flume using a peristaltic pump (model: CT1000; Baoding Chuangrui Precision Pump Co., Ltd., Hebei Province. China), a water inlet pipe (material: rigid PVC pipe; inner diameter: 2.0 cm) and a valve. The barreled experimental water in the laboratory can be directly fed into the experimental flume by the peristaltic pump and a PU hose (inner diameter: 2.0 cm). In a case that the water in the experimental flume exceeds an overflow weir, the water may be fed into a water return tank below through an overflow pipe. In a case that the water surface in the water return tank exceeds a submersible pump, the submersible pump may be turned on to pump the water from the water return tank to the experimental flume through a return pipe. The water inlet of the experimental flume is stopped when the liquid level approaches the height of the water return tank. The volume of a plastic round barrel for containing the experimental water is 100 L. The experimental water may also be prepared in the experimental flume: a nozzle of laboratory tap water is connected to that of the main water inlet pipe, the tap water is directly fed into the experimental flume and the water return tank through the main water inlet pipe, the water inlet pipe, the peristaltic pump and the valve. After the water inlet operation is stopped, a dosing device is turned on by an industrial personal computer of a control cabinet. Under the action of the submersible pump, the overflow pipe and the return pipe, the acid/alkali or reagent can circulate between the water return tank and the experimental flume for many times to be mixed evenly. The PU hose can be used to feed the barreled experimental water into the water inlet tank and its water return tank, and then the water in the water inlet tank can be fed into the experimental flume by using the above method.

Wave-making: the industrial personal computer of the control cabinet can be used to turn on a wave-making system, and adjust the rotating speed of a wave maker (0 rpm to 100 rpm) according to the experimental requirements, thus making the water body have waves similar to those of lakes.

Illumination: the industrial personal computer is configured to turn on an illumination lamp switch, to set solar illumination with 24-hour regular illumination, where the change of sunlight above the experimental flume in one day is consistent with that of the lake, or a fixed illumination intensity is input into the industrial personal computer.

Turn on environmental condition parameter online monitor: The heights of environmental parameter sensor probes (pH/Eh/DO/conductivity) are adjusted through a scale holder made of aluminum alloy and a stainless-steel sensor holder, thus controlling the depths of the monitoring probes in the water body layer. Four environmental parameter data and data change curves along with the time are displayed on a display screen of the industrial personal computer, and the data can be saved in real time. In order to research spatial distribution characteristics of environmental parameters at 100 μm spatial scale at the sediment/water interface, microelectrodes or planar optodes (PO) can be used to test at the interface, thus obtaining a one-dimensional vertical distribution image of pH/Eh/DO or a two-dimensional spatial distribution image of pH/DO.

Turn on water quality index analyzer: A total phosphorous/total nitrogen online analyzer, an ammonia nitrogen online analyzer and a COD_{Cr} online analyzer are connected to a data acquisition module and a PLC of the control cabinet through signal cables, and power supply lines are connected to a switch of the control cabinet. Measurement and calibration parameters are set on a display screen page of the industrial personal computer, and then a button of “water quality index analyzer module” is clicked to determine test modes of the four experimental flumes; one or more experimental flumes as well as a “single test” or “cycle test” mode can be selected. In this way, the four water quality indexes of the experimental flumes can be determined once or many times in one day, and a maximum of 12 cycles of determination per day can be set. The environmental condition parameters, water quality indexes and other data curves changing with the time of each experimental flume can be automatically displayed on the display screen of the industrial personal computer, and the data can be saved in the industrial personal computer.

Turn on water automatic sampling device: Two water automatic sampling devices are configured to collect water from the experimental flumes or the water return tanks through micro solenoid valves and sampling pipelines (four paths) of the sampler; a signal cable of the solenoid valve and a control line of the sampler are connected to the data acquisition module, switch and PLC of the control cabinet. The control cabinet can be used to set the “single sampling” or “circular sampling”, so as to realize single or multiple sampling within one day, and a maximum of 12 cycles a day can be set to collect water samples, with the sampling volume from 50 ml to 400 ml each time. A freezer of the automatic sampling device can be used to preserve twenty-four bottles of water samples at 4° C. The offline monitor is used for subsequent analysis and testing, so as to analyze nitrogen, phosphorus, heavy metals and organic matters in the water sample.

Control of environmental conditions (aeration or dosing): One of three modes (air, argon gas, and pure oxygen) of the aeration device may be selected according to the DO target value of the simulation experiment in water body and DO online data fed back to the industrial personal computer by the environmental condition parameter online monitor. The industrial personal computer is used to set a DO target value, and then the aeration system is turned on. The aeration head automatically stops aeration when the DO displayed by the industrial personal computer online reaches the set value. According to pH target value of the simulation experiment in the water body and pH online data fed back to the industrial personal computer online, the industrial personal computer can select whether to add acid (HCl) or alkaline solution (NaOH) and dosing amount, set the pH target value, and then turn on the dosing device. The dosing is automatically stopped when the pH displayed by the industrial personal computer online reaches the set value. If reagents are required to add in the simulation experiment, the dosing time of “pollutants” is set by the industrial personal computer, and the dosing is automatically finished when the dosing amount reaches a target value.

Simulation experiment: After the water environment conditions, illumination, aeration, wind and wave conditions and the operations of various online testers and water samplers are stable, the sediment/water interface stratification is stable, and aquatic plants grow up and meet the requirements, the simulation experiment can be carried out, for example: (1) an experiment on a transfer-release mechanism of nitrogen and phosphorus in sediments; (2) an experiment for research of nitrogen and phosphorus process in the rhizosphere of submerged plants and the mechanism of phytoextraction; or (3) an experiment of sediment passivation and research on ecological restoration mechanism for nitrogen and phosphorus. The required experiments or started devices include: an automatic dosing or aeration device for controlling pH or DO of the water body; an automatic dosing for adding a water purification reagent into the water body or artificially adding passivator to the surface layer of the sediment; an illumination or wave-making device for controlling the illumination or hydraulic disturbance conditions; an environmental condition parameter online monitor and online water quality index analyzer for determining water environmental parameters and water quality indexes; a water automatic sampling device for the collection and preservation of water samples; and an offline monitor for determining the water samples (nitrogen and phosphorus forms; heavy metal elements; organic matters). The industrial personal computer is used to save above online data and study the biological effectiveness of nitrogen and phosphorus in the water body and at sediment/water interface and the their cycle characteristics at interface. Samples of the sediments and aquatic plants are collected manually, and the total amount and forms of nitrogen, phosphorus and heavy metals, organic matter content, aquatic plant tissue weight, element content and root surface area in sediments are analyzed. The sediments or aquatic plant rhizosphere can also be tested in situ (DGT, Peeper, microelectrodes, planar optodes), and can be subsequently analyzed and tested to obtain one-dimensional or two-dimensional high spatial resolution distributions (100 μm to mm) of elements or environmental condition parameters in the sediments or aquatic plant rhizosphere. By combining online parameters, water quality indexes and offline analyzed physical-chemical properties indexes, the formation mechanism of internal loading of nitrogen and phosphorous in the sediments, the transfer-transform process of nitrogen and phosphorus in aquatic plant rhizosphere, the passivation mechanism for the sediments and technical processes are revealed under different environmental conditions.

Stop the experiment: After above simulation experiments are finished, the in-situ testing devices (DGT. Peeper, microelectrodes, planar optodes) at the sediment/water interface are taken out, the industrial personal computer of the control cabinet is used to turn off the wave-making device, the illumination device, the submersible pump in the water return tank, the environmental condition parameter online monitor, the online water quality index analyzer and the water automatic sampling device, so as to make the above devices stand by. Then, the environmental parameter sensor probes are taken out from the water using the scale holder made of aluminum alloy; and electrodes are placed in protective solution.

Drainage: The water in the experimental flume may be drained into the sewer through the peristaltic pump, the valve, the drainage branch pipe, and the main drainage pipe. The water in the experimental flume may also be introduced into the water inlet tank for preservation through the peristaltic pump, the valve, the drainage branch pipe, the main water inlet pipe, and the water inlet branch pipe. When there is no water in the experimental flume, the peristaltic pump is turned off and the valve is closed to finish the drainage. The water in the water return tank may be drained into the sewer or the water inlet tank through the peristaltic pump and the PU hose. If the water in the water inlet tank and its water return tank is required to be drained, the water can be drained into the sewer through the peristaltic pump and the PU hose.

Sludge discharge: A small amount of water can be added to the sediment at the lower part of the experimental flume, and after the water and the sediment are manually stirred into slurry, a sludge discharge pipe of the experimental flume is opened to discharge the slurry into a sludge storage barrel; or the sediment in the experimental flume can be directly pumped out through a sludge discharge pump and the PU hose and then discharged into the sludge storage barrel. Finally, the water is used to flush the experimental flume, and the above operation is used to drain the water-sludge mixture from the experimental flume into the sludge storage barrel.

Preparation for next simulation experiment: a simulation system is restarted for a new experiment according to above steps.

Particularly, in a case that simulation experiments of two or more experimental flumes are required to carry out, four operations of “adding sediment, adding water, making wave and turning on the environmental condition parameter online monitor” can be firstly carried out on the first experimental flume, and then the above operations are adopted for the second or more experimental flumes. After the operations on all experimental flumes are finished, the water quality index analyzer and the water automatic sampling device are turned on in turn to control the environmental conditions (aeration or dosing); and simulation experiments of all experimental tanks are respectively carried out after the sediment/water interfaces in the experimental flumes are stabilized. When the simulation experiment in a certain experimental flume is finished, the operations of “stopping experiment, draining water and discharging sludge”, are carried out, and then the operations of “stopping experiment, draining water and discharging sludge” in other experimental flumes are carried out in turn. In a case that the experiments in other experimental flumes are not finished, the experiment can be continued. Note: When the above experimental flume is subjected to the operations of “adding water and draining water”, the valves on the main drainage pipe and the main water inlet pipe should be correctly opened or closed, ensuring that the water cannot be fed into other experimental flumes or the water inlet tank.

### Embodiment 2

Based on the simulation device of this embodiment, an experiment corresponding to “study on evaluation of transfer-release mechanism and internal loading of ammonia nitrogen/nitrate nitrogen/phosphorus in lake anoxic sediments” can be carried out. The experiments mainly include: (1) regulating environmental conditions of a sediment/water interface by a simulation device; (2) measuring the sediment/water interface based on DGT (diffusive gradients in thin films) and a Peeper device; (3) evaluating transfer-release mechanism and internal loading of ammonia nitrogen/nitrate nitrogen/phosphorus in lake anoxic sediments, specifically including the following steps:

(1) Collection of sediment and water samples: Sediments and water used in the simulation experiment are collected from Dalai Nur Lake, Inner Mongolia Autonomous Region, China. On a sampling ship, a grab sampler is used to collect 200 L of sediments from Daley Lake, and a bucket is used to collect 800 L of water. Then, the sediment and water samples are respectively packed into round plastic barrels of 50 L or 100 L, and transported to a laboratory for analyzing and testing the physical-chemical properties of nitrogen, phosphorus and heavy metals in the sediment and water samples. The barreled experimental water is introduced into a water inlet tank through a peristaltic pump and a PU hose. When the water level (98 cm) reaches above an overflow weir of the water inlet tank, and the water is introduced into a water return tank through an overflow pipe. When the water reaches the liquid level in the water return tank, the submersible pump is turned on the water circulation between the water inlet tank and the water return tank is achieved through the overflow pipe and the overflow pipe, and the introduction of the barreled water to the water inlet tank is stopped.

(2) Adding of sediments: The collected wet sediments are poured into one experimental flume from the upper part of the experimental flume; a small amount of water (5 L) is added to moist the sediments after the sediments are laid every 5 cm, the sediments are kept flat as much as possible. The sediments are laid for a total of four times, with a total of 192 L of sediments used. The final sediment thickness is about 20 cm.

(3) Adding of water: After the sediments are added, the water in the water inlet tank is fed into the experimental flume through the water inlet pipe, the peristaltic pump and the valve, and the flow velocity is kept at 4 L/min. A total of 768 L of water is injected into the experimental flume, with a thickness of 92 cm. When the water level exceeds the overflow wire and the water in the water return tank below reaches the liquid level, the submersible pump is turned on to achieve water circulation between the experimental flume and the water return tank. At the moment, the water is continuously added, and the water introduction is stopped when the water level in the experimental flume reaches 98 cm.

(4) Turn on automatic control module, turn on wave-making system and illumination system: After the sediment/water interface is stabilized, the automatic control module is turned on, a schematic diagram and a parameter box of the simulation device are displayed on the industrial personal computer; and then the wave-making device is turned on by the industrial personal computer to form certain waves on the surface water in the experimental flume. Meanwhile, the automatically changed sunlight with 24-hour by the illumination device is set to make the change of sunlight above the experimental flume within one day consistent with that of the natural lake.

(5) Turn on environmental condition parameter online monitor: The heights of environmental parameter sensor probes (pH/Eh/DO/conductivity) are adjusted through a scale holder made of aluminum alloy and a stainless-steel sensor holder, thus controlling the depths (40 cm) of the monitoring probes in the water body layer. The environmental condition parameter online monitor is turned on by the industrial personal computer to monitor water environmental parameters (pH/Eh/DO/conductivity), and the data are transmitted to the industrial personal computer. The change curves of environmental parameters every 15 minutes are displayed on a screen of the industrial personal computer in real time. When the above four environmental parameters remain stable for three consecutive days (RSD<5%; n=15; fifteen data collected at the same interval), the following experiments can be carried out. Prior to the simulation experiment in the experimental flume, the water body is stabilized and pH=8.0. DO=8.5 mg/L, Eh=231.1 mv, and conductivity=322.8 μs/s.

(6) Turn on online water quality index analyzer: A NPW160 total nitrogen/total phosphorus online analyzer, a COD online analyzer and an AmtaxSC ammonia nitrogen online analyzer are turned on by the industrial personal computer, and are set to sample and determine twice a day and to analyze once every 12 hours. The industrial personal computer is used to collect the data and to display a water quality index curve changing with time online.

(7) Turn on water automatic sampling device: The water automatic sampling device is turned on by the industrial personal computer and is set to sample twice a day and analyze once every 12 hours. Twenty-four bottles of water samples can be preserved in a refrigerator inside each water automatic sampling device. Excess water samples are transferred and preserved in the laboratory refrigerator.

(8) Control of environmental conditions (aeration+dosing): Based on the water environmental condition parameters determined by the environmental condition parameter online monitor and the experimental requirements, the target values of water environmental conditions are adjusted as follows: pH=8.50 and DO=2.2 mg/L. The method is as follows: turning on aeration and dosing devices by the industrial personal computer. In the aeration device, argon gas is aerated to carry out anoxic treatment of water body until the DO reaches 2.2 mg/L. In the dosing device, alkaline solution (NaOH) is added to alkalize the water body until the pH reaches 8.50. The environmental condition parameter online monitor is used to monitor pH/DO and other parameters of middle water in real time, and to feed the parameters back to the industrial personal computer in real time. When DO or pH reaches the target value, the industrial computer immediately stops the operation of the aeration and dosing devices. If DO or pH deviates from the target value, argon or pure oxygen is continuously aerated (DO is higher or lower than 2.2 mg/L); or alkaline solution (NaOH) or acid solution (HCl) (pH below or above 8.50) is added until the DO or pH reaches the target value.

(9) Simulation experiment: The environmental parameters of middle water are determined by the environmental condition parameter online monitor (monitoring once every 15 minutes), and the four indexes of the middle water are analyzed by the water quality index online analyzer (twice a day). The water automatic sampling device and the offline monitor are used to analyze other water quality indexes of surface water (twice a day); and then the above data are analyzed consecutively for five days. When the RSD of various parameters are less than 5% (n=7), the environmental conditions and nitrogen and phosphorus cycles at the sediment/water interface reach a stable state. Therefore, in-situ DGT test of ammonia nitrogen, nitrate nitrogen and phosphorus at the sediment/water interface can be carried out.

Experiment for transfer-release mechanisms of ammonia nitrogen, nitrate nitrogen and phosphorus is as follows: two double-sided DGT probes: AMP-TH&ZrO-chelex and Zr-oxide & AgI DGT and a HR-Peeper probe are inserted vertically into the sediment/water interface with a horizontal distance of 2 cm. The windows of DGT probe and Peeper at the overlying water part are 2 cm or 5 cm, and the sediment is 13 cm or 15 cm, respectively. The test time of the DGT and HR-Peeper probes is 24 h or 48 h. respectively. Then DGT probe and HR-Peeper are retrieved for subsequent analysis. Meanwhile, the sediment core for the test by the DGT and HR-Peeper are collected by a columnar sediment sampler and the sediment core. DGT and HR-Peeper undergo subsequent analyses.

(10) Subsequent analyses of DGT probe and HR-Peeper: AMP-TH. ZrO-chelex, Zr-oxide and AgI resins of the two double-sided DGT probes are taken out. AMP-TH or ZrO-chelex are cut by a ceramic row cutter with a vertical resolution of 2 mm. A gel strip is put into a centrifugal test tube and eluted with acid or alkali solution, and a microplate spectrophotometer is used for analysis: phosphorus, ammonia nitrogen and nitrate nitrogen are analyzed by phosphorus molybdenum blue spectrophotometry, alicylic acid-hypochlorite colorimetry, and ultraviolet spectrophotometry, respectively, and one-dimensional DGT concentration profiles of ammonia nitrogen, nitrate nitrogen and phosphorus are derived. After the Zr-oxide and AgI resins are dried, the images are scanned by a flat scanner, and two-dimensional spatial distribution images of DGT concentrations in the phosphorus or sulfur are calculated by computer imaging densitometry (CID), and then the DGT-phosphorus and DGT-sulfur are converted into two-dimensional distribution images with the same horizontal direction. The solution from each chamber of HR-Peeper (400 μL) is extracted into the centrifuge test tube and preserved with 20 μL of hydrochloric acid (0.1 mol/L). The microplate spectrophotometer is still used for analysis: phosphorus, ammonia nitrogen, nitrate nitrogen and Fe (II) are analyzed by phosphorus molybdenum blue spectrophotometry, alicylic acid-hypochlorite colorimetry, ultraviolet spectrophotometry and phenanthroline spectrophotometry, respectively, and one-dimensional profiles of ammonia nitrogen, nitrate nitrogen, iron and phosphorus concentrations in sediment porewater and two-dimensional images of sulfur and phosphorus are drawn based on the analyses of DGT and peeper.

(11) Test of columnar sediments: A rubber plug at the side wall of the PVC pipe is opened after the sediment core is taken out, the pH/DO of the columnar sediment (0 cm to −15 cm) is tested by using a microelectrode probe (Presens company, Germany) with a vertical spatial resolution of 1.0 cm, thus obtaining DO profiles of the sediment porewater. The sediment columnar sample (0 cm to 15 cm) is divided into 15 segments, dried, ground, and screened by 100 μm, and then the total amount and speciation content of nitrogen, phosphorus and heavy metals (iron, aluminum and calcium) are determined, and the organic matters are analyzed at the same time.

(12) Study on geochemical processes of nitrogen, nitrate and phosphorus: Based on the DGT concentration profiles of ammonia nitrogen/nitrate nitrogen/phosphorus/Fe (II) and their porewater profiles (Peeper), the resupply parameter (R) profiles of the above elements in sediments are obtained; the resupply capacity of N and P in sediment solid phase is evaluated by combining the abovementioned profiles of R values, the DO profile of the sediment and the physical-chemical properties of the sediment. By comparing and analyzing the DGT profiles of ammonia nitrogen/nitrate nitrogen/phosphorus/iron and DO profiles or Fe—Al—Ca organic matters in sediments, the transfer-release mechanisms, geochemical process and formation mechanism of ammonia nitrogen/nitrate nitrogen/phosphorus, and an influence of Fe—Al—Ca organic matters on the release of ammonia nitrogen/nitrate nitrogen/phosphorus in anoxic sediments are revealed. Meanwhile, the DGT two-dimensional images of phosphorus and sulfur and DGT-iron profile are analyzed, and the spatial variation characteristics of phosphorus, iron and sulfur are compared to study the phosphorus release caused by the coupled geochemical reactions of the three elements. The distribution characteristics of phosphorus microniches in sediments is analyzed to study a formation mechanism of the phosphorus microniches in the anoxic sediments.

### (13) Estimation and Evaluation of Internal Loading of Ammonia Nitrogen, Nitrate Nitrogen and Phosphorus

Based on the DGT profiles of sediment/overlying water with ammonia nitrogen, nitrate nitrogen, nitrogen and phosphorus, and the following calculation formulas (1-2).

The diffusion flux and internal loading of nutrient elements at the sediment/water interface can be estimated.

$$F = F s + F w = ( - ↓ D s \frac{{C}_{s}}{{X}_{s}} ) + ( - D w \frac{{C}_{w}}{{X}_{w}} ) \qquad Formula 1$$

$$W = F \times A \times t \qquad Formula 2$$

F is the apparent diffusion flux (μg m⁻² d or mg m⁻² d) in the sediment/water interface; Fs and Fw are diffusion fluxes (μg m⁻² d or mg m⁻² d) in the surface sediment and overlying water layer; and a diffusion coefficient Ds (cm s⁻¹) of the sediment is derived from the calculation of molecular diffusion flux in water and sediment porosity (ϕ). Cₛ/ [symbol] Xₛ and [symbol] C_{w}/X_{w} are respectively diffusive gradients of the surface sediment and the overlying water layer. W (μg a⁻¹) is the internal loading of ammonia nitrogen, nitrate nitrogen, nitrogen or phosphorus in the sediment in the simulation device; A is the area (m²) of the sediment; and t is 365 days of the year.

It should be noted that: for those skilled in the art, apparently, the present disclosure is not limited to details of the exemplary embodiments, and may be expressed in other specific forms without departing from the spirit or basic characteristics of the present disclosure. Therefore, in any way, the embodiments should be regarded as exemplary, not limitative; and the scope of the present disclosure is limited by the appended claims, instead of the above description. Thus, all variations intended to fall into the meaning and scope of equivalent elements of the claims should be covered within the present disclosure. Any reference signs in the claims shall not be regarded as limitations to the concerned claims.

Several examples are used for illustration of the principles and implementation methods of the present disclosure. The description of the embodiments is merely used to help illustrate the method and its core principles of the present disclosure. In addition, those of ordinary skill in the art can make various modifications in terms of specific embodiments and scope of application in accordance with the teachings of the present disclosure. In conclusion, the content of this specification shall not be construed as a limitation to the present disclosure.

1. An intelligent simulation device for bottom sediment pollution process and control, comprising:
an experimental flume, which is able to be used for simulating nitrogen and phosphorus processes at a sediment/water interface, wherein a water inlet tank and a water return tank are connected to the experimental flume, the water inlet tank is used to preserve experimental water and to add water to the experimental flume; the water return tank is connected to the experimental flume, and is used to achieve the return between water body of the experimental flume and a water body of the water return tank;
a wave-making system, which is installed on an upper part of an inner wall of the experimental flume and is used to produce wind waves on the sediment/water interface;
an illumination system, which is installed directly above the experimental flume and is used to simulate illumination;
a dosing system, which is installed below the experimental flume and is used to add experimental reagents into the experimental flume to control pH of a water body in the experimental flume, or add pollutants or flocculants;
an aeration system, which is installed on the upper part of the inner wall of the experimental flume, and is used to perform aeration treatment on the water body in the experimental flume, so as to control dissolved oxygen of the water body in the experimental flume;
an environmental condition parameter online monitor, which is installed on the inner wall of the experimental flume and is able to extend to different depths of the water body in the experimental flume for online determination of water body environmental parameters, such as pH, Eh, DO, and conductivity;
an online water quality index analyzer, which is used to monitor water quality indexes in real time, comprising total nitrogen, ammonia nitrogen, total phosphorus, and COD_{Cr};
a water automatic sampling device, which is connected to the experimental flume or the water return tank, and is able to automatically collect water samples in a single or cycle mode;
an offline analyzer, which is used to determine water quality indexes of the collected water samples or physical-chemical indexes of sediments; and
an automatic control module, wherein the wave-making system, the illumination system, the dosing system, the aeration system, the environmental condition parameter online monitor, the online water quality index analyzer, the water automatic sampling device and the offline analyzer are all connected to the automatic control module, and split devices are directed by the automatic control module to operate according to set parameters and collect data online.

2. The intelligent simulation device for bottom sediment pollution process and control according to claim 1, wherein the experimental flume and the water inlet tank are both installed on a support, and a support cavity is provided below the support; the support cavity is used to accommodate experimental devices; the experimental devices comprise a peristaltic pump, a valve, a pipeline, the water return tank, a solenoid valve, a sampling cup, and a tee; and the experimental flume is connected to one water return tank through a return pipe, the experimental flume is further provided with an overflow weir, and the overflow weir is connected to the water return tank through an overflow pipe.

3. The intelligent simulation device for bottom sediment pollution process and control according to claim 2, wherein four experimental flumes and four water inlet tanks are correspondingly provided; the four experimental flumes are able to operate independently; or, after the four experimental flumes are connected in series, the water body is able to circulate.

4. The intelligent simulation device for bottom sediment pollution process and control according to claim 1, wherein a main device of the wave-making system is a wave maker, which is fixed onto the inner wall of the experimental flume and is able to perform height adjustment in surface water; and
a main device of the illumination system is an illumination lamp, which has a color temperature of 400 k, a power of 150 W and an illumination intensity from 0 Lux to 10,000 Lux; the illumination lamp is able to automatically simulate the change of sunlight within 24 hours a day, and the illumination intensity of the illumination lamp is able to be fixed.

5. The intelligent simulation device for bottom sediment pollution process and control according to claim 2, wherein the dosing system comprises reagent bottles, the number of the reagent bottles is three, the three reagent bottles are respectively used to place acid, alkali, and chemical agent; the reagent bottles each are connected to the water return tank by a dosing pipeline, and a metering pump is connected to the dosing pipeline; and
the aeration system comprises an aeration head, the aeration head is connected to a gas supply system through a gas path, an air pump is also connected to the gas path, and the gas path is provided with a flowmeter, a control valve, and a pressure relief valve.

6. The intelligent simulation device for bottom sediment pollution process and control according to claim 2, wherein the environmental condition parameter online monitor comprises monitoring probes; the monitoring probes comprise a pH electrode, a fluorescence dissolved oxygen sensor, a conductivity electrode, and an Eh electrode; and the monitoring probes are fixed onto the inner wall of the experimental flume, and depths of the monitoring probes in the water body are able to be adjusted through a scale holder made of aluminum alloy.

7. The intelligent simulation device for bottom sediment pollution process and control according to claim 1, wherein the online water quality index analyzer comprises an ammonia nitrogen automatic analyzer, a total phosphorus/total nitrogen automatic analyzer, and a COD_{Cr} automatic analyzer; and the online water quality index analyzer is connected to the experimental flume.

8. The intelligent simulation device for bottom sediment pollution process and control according to claim 1, wherein the number of the water automatic sampling devices is two, a refrigerator type sampler is used as the water automatic sampling device, and the water automatic sampling device is connected to the water body in the water return tank or the experimental flume; and
the offline analyzer comprises a multifunctional microplate spectrophotometer, a GC/MS analyzer, an ICP-MS analyzer, a laser particle size analyzer, a Unisense microelectrode, and a planar optode system.

9. The intelligent simulation device for bottom sediment pollution process and control according to claim 1, wherein a PLC is used as the automatic control module, the PLC is a programmable logic controller control system, and the automatic control module is installed in a control cabinet; and a touch screen page of an industrial personal computer of the automatic control module is capable of displaying a structure and operation status of the intelligent simulation device, and the online monitoring number, change curves of environmental parameters and water quality indexes along with time in real time.

10. An experimental method based on the intelligent simulation device for bottom sediment pollution process and control according to claim 1, comprising the following steps:
collecting sediment and water samples;
adding the sediments to an experimental flume;
adding the water samples to the experimental flume;
turning on an automatic control module, a wave-making system and an illumination system;
turning on an environmental condition parameter online monitor for online determination of environmental parameters in water body;
turning on an online water quality index analyzer for real-time monitoring of water quality indexes;
turning on a water automatic sampling device;
controlling environmental conditions;
starting a simulation experiment;
after the simulation experiment is completed, stopping the experiment; draining water and discharging sediments; and
preparing for the next simulation experiment.

11. The experimental method according to claim 10, wherein the experimental flume and the water inlet tank are both installed on a support, and a support cavity is provided below the support; the support cavity is used to accommodate experimental devices; the experimental devices comprise a peristaltic pump, a valve, a pipeline, a water return tank, a solenoid valve, a sampling cup, and a tee; and the experimental flume is connected to one water return tank through a return pipe, the experimental flume is further provided with an overflow weir, and the overflow weir is connected to the water return tank through an overflow pipe.

12. The experimental method according to claim 11, wherein four experimental flumes and four water inlet tanks are correspondingly provided; the four experimental flumes are able to operate independently; or, after the four experimental flumes are connected in series, the water body is able to circulate.

13. The experimental method according to claim 10, wherein a main device of the wave-making system is a wave maker, which is fixed onto the inner wall of the experimental flume and is able to perform height adjustment in the surface water; and
a main device of the illumination system is an illumination lamp, which has a color temperature of 400 k, a power of 150 W and an illumination intensity from 0 Lux to 10,000 Lux; the illumination lamp is able to automatically simulate the change of sunlight within 24 hours a day, and the illumination intensity of the illumination lamp is able to be fixed.

14. The experimental method according to claim 11, wherein the dosing system comprises reagent bottles, the number of the reagent bottles is three, the three reagent bottles are respectively used to place acid, alkali and chemical agent; the reagent bottles each are connected to the water return tank by a dosing pipeline, and a metering pump is connected to the dosing pipeline; and
the aeration system comprises an aeration head, the aeration head is connected to a gas supply system through a gas path, an air pump is also connected to the gas path, and the gas path is provided with a flowmeter, a control valve, and a pressure relief valve.

15. The experimental method according to claim 11, wherein the environmental condition parameter online monitor comprises monitoring probes; the monitoring probes comprise a pH electrode, a fluorescence dissolved oxygen sensor, a conductivity electrode, and an Eh electrode; and the monitoring probes are fixed onto the inner wall of the experimental flume, and depths of the monitoring probes in the water body are able to be adjusted through a scale holder made of aluminum alloy.

16. The experimental method according to claim 10, wherein the online water quality index analyzer comprises an ammonia nitrogen automatic analyzer, a total phosphorus/total nitrogen automatic analyzer, and a COD_{Cr} automatic analyzer; and the online water quality index analyzer is connected to the experimental flume.

17. The experimental method according to claim 10, wherein the number of the water automatic sampling devices is two, a refrigerator type sampler is used as the water automatic sampling device, and the water automatic sampling device is connected to the water body in the water return tank or the experimental flume; and
the offline analyzer comprises a multifunctional microplate spectrophotometer, a GC/MS analyzer, an ICP-MS analyzer, a laser particle size analyzer, a Unisense microelectrode, and a planar optode system.

18. The experimental method according to claim 10, wherein a PLC is used as the automatic control module, the PLC is a programmable logic controller control system, and the automatic control module is installed in a control cabinet; and a touch screen page of an industrial personal computer of the automatic control module is capable of displaying the structure and operation status of the intelligent simulation device, and the online monitoring number, change curves of environmental parameters and water quality indexes along with time in real time.
